(19) United States
(12) Patent Application Publication
(10) Pub. No.: US 2019/0313941 A1
(43) Pub. Date: Oct. 17, 2019

(54) METHODS, SYSTEMS AND APPARATUSES FOR DISPLAYING REAL-TIME CATHETER POSITION

(71) Applicant: AVINGER, INC., Redwood City, CA (US)

(72) Inventor: Ryan RADJABI, Campbell, CA (US)

(21) Appl. No.: 16/347,840

(22) PCT Filed: Nov. 16, 2017
(86) PCT No.: PCT/US17/62006

§ 371 Date: May 7, 2019

Related U.S. Application Data
(60) Provisional application No. 62/423,064, filed on Nov. 16, 2016.

(51) Int. Cl. A61B 5/06; A61B 5/00; A61B 6/12; A61B 6/00; A61B 34/20

(52) U.S. Cl. CPC A61B 5/066; A61B 5/0066; A61B 6/12; A61B 6/487; A61M 2025/0166; A61B 5/0084; A61B 6/463; A61B 34/20; A61B 6/5247

(57) ABSTRACT

A system for tracking and displaying a real-time catheter position overlaying a fluoroscopic image includes a catheter having an optical coherence tomography (OCT) sensor thereon, a displacement sensor configured to identify displacement of the catheter, a controller, and a display. The controller is configured to receive a fluoroscopic image of the distal end of the catheter at a first position and determine a displacement of the catheter from the first position to a second position using the displacement sensor. The display is configured to display the distal end of catheter at the second position as an overlay on the fluoroscopic image.

### CROSS REFERENCE TO RELATED APPLICATIONS

This application claims priority to U.S. Provisional Application No. 62/423,064, filed Nov. 16, 2016, entitled “METHODS, SYSTEMS AND APPARATUSES FOR DISPLAYING REAL-TIME CATHETER POSITION”, the entirety of which is incorporated by reference herein.

This application may be related to U.S. patent application Ser. Nos. 13/939,161 and 14/171,583, the entireties of which are incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Peripheral artery disease (PAD) and coronary artery disease (CAD) affect millions of people in the United States alone. PAD and CAD are silent, dangerous diseases that can have catastrophic consequences when left untreated. CAD is the leading cause of death in the United States while PAD is the leading cause of amputation in patients over 50 and is responsible for approximately 160,000 amputations in the United States each year.

Coronary artery disease (CAD) and Peripheral artery disease (PAD) are both caused by the progressive narrowing of the blood vessels most often caused by atherosclerosis, the collection of plaque or a fatty substance along the inner lining of the artery wall. Over time, this substance hardens and thickens, which can cause an occlusion in the artery, completely or partially restricting flow through the artery. Blood circulation to the arms, legs, stomach and kidneys brain and heart may be reduced, increasing the risk for stroke and heart disease.

Interventional treatments for CAD and PAD may include endarterectomy and/or atherectomy. Endarterectomy is surgical removal of plaque from the blocked artery to restore or improve blood flow. Endovascular therapies such as atherectomy are typically minimally invasive techniques that open or widen arteries that have become narrowed or blocked. Often, occlusion-crossing devices can be used to ease the passage of such devices through a blockage.

Minimally invasive techniques can be enhanced through the use of on-board imaging, such as optical coherence tomography (“OCT”) imaging. However, while on-board imaging be beneficial in visualizing the tissue as the catheter travels therethrough, it cannot be used to show the relative placement of the catheter within the body (e.g., within the blood vessel).

Identification of the real-time position of the catheter relative to the patient's anatomy would be beneficial to surgeons during interventional treatments for more efficient and accurate treatment. Fluoroscopy remains the cornerstone of imaging the relative placement of the catheter within the lumen of the vessel in most interventional procedures. Prolonged exposure to fluoroscopy, however, increases radiation exposure, both for the patients and the physicians. As a result, the patients may have an increased risk of lifetime malignancy. The physicians are also exposed to increasing radiation hazards. Recent reports have, for instance, revealed that there may be an excess risk of brain tumors among interventional cardiologists.

There is thus a pressing need to develop methods and systems for reducing radiation during minimally invasive procedures while still providing an accurate position of the catheter relative to a patient's body in interventional procedures.

### SUMMARY OF THE DISCLOSURE

In general, in one embodiment, a system for tracking and displaying a real-time catheter position overlaying a fluoroscopic image includes a catheter having an optical coherence tomography (OCT) sensor thereon, a displacement sensor configured to identify displacement of the catheter, a controller, and a display. The controller is configured to receive a fluoroscopic image of the distal end of the catheter at a first position and determine a displacement of the catheter from the first position to a second position using the displacement sensor. The display is configured to display the distal end of catheter at the second position as an overlay on the fluoroscopic image.

This and other embodiments can include one or more of the following features. The display can be further configured to display an OCT image from the OCT sensor. The fluoroscopic image can be a static fluoroscopic image. The controller can be further configured to synchronize a zero position of the distal end of the catheter with the first position when the fluoroscopic image can be captured. Determining the displacement of the catheter can include determining the location of the distal end of the catheter at the second position using signals from the displacement sensor. The displacement sensor can be attached to the catheter. The displacement sensor can be axially movable relative to the catheter. The displacement sensor can be a separate component from the catheter. The displacement sensor can be an optical sensor. The displacement sensor can be a mechanical sensor. The displacement sensor can be an electromagnetic positioning sensor. The catheter can be an atherectomy catheter. The catheter can be an occlusion-crossing catheter.

In general, in one embodiment, a method for tracking and displaying a real-time catheter position includes inserting a catheter into a body lumen, capturing an optical coherence tomography (OCT) image with an OCT sensor on the catheter, capturing a fluoroscopic image of the distal end of the catheter at a first position, displaying the fluoroscopic image on a display, advancing the catheter to a second position, determining a displacement of the catheter from the first position to the second position using a displacement sensor, and displaying the distal end of catheter at the second position as an overlay on the fluoroscopic image on the display.

This and other embodiments can include one or more of the following features. Capturing a fluoroscopic image can include capturing a static fluoroscopic image, and displaying can include displaying the static fluoroscopic image. The method can further include synchronizing a zero position of the distal end of the catheter with the first position when the fluoroscopic image is captured. The method can further include displaying the zero position of the distal end of the catheter on the fluoroscopic image. Determining the displacement of the catheter can include determining the location of the distal end of the catheter at the second position using signals from the displacement sensor. The method can further include displaying the OCT image. The displacement sensor can be attached to the catheter. The displacement sensor can be axially movable relative to the catheter. The method can further include placing the displacement sensor at an insertion point of the catheter into the body lumen. The method can further include reducing an overall amount of x-ray radiation by only capturing a single fluoroscopic image for a travel range of the catheter that can be displayed within a view of the single fluoroscopic image. The displacement sensor can be an optical sensor. The displacement sensor can be a mechanical sensor. The displacement sensor can be an electromagnetic positioning sensor.

In general, in one embodiment, a catheter device includes an elongate body extending from a proximal end to a distal end, an optical coherence tomography (OCT) sensor attached to the elongate body, and a displacement sensor attached to the elongate body and axially movable relative to the movable body. The displacement sensor is configured to measure a displacement of the distal end of the catheter relative to the displacement sensor. The displacement sensor is configured to be connected to a controller configured to receive signals from the displacement sensor and determine a position of the distal end.

This and other embodiments can include one or more of the following features. The displacement sensor can be an optical sensor. The displacement sensor can be a mechanical sensor. The displacement sensor can be an electromagnetic positioning sensor. The catheter can be an atherectomy catheter. The catheter can be an occlusion-crossing catheter.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a side perspective view of an exemplary catheter device including a sensor to detect displacement of the catheter.

FIGS. 2A and 2B show OCT and fluoroscopy screen captures of an exemplary catheter device.

FIGS. 3A and 3B show the orientation of an OCT image and a corresponding fluoroscopy image from an OCT catheter device.

FIGS. 4A-4C show OCT and fluoroscopy screen captures used to aid steering an exemplary catheter device.

FIG. 5 illustrates a block diagram of a system configured to track and display a real time position of a catheter according to one embodiment of the disclosure.

FIGS. 6A-6C show a catheter at a “zero” position.

FIG. 7 schematically illustrates inputting data regarding the “zero” position of a catheter into a processor.

FIGS. 8A-8B show a catheter at a second, non-zero position.

FIGS. 9A-9C illustrate various positions of a catheter as displaced on a static image.

FIG. 10 is a flow diagram of a method for tracking and displaying a real-time catheter position.

### DETAILED DESCRIPTION

Described herein are methods, systems and apparatuses for tracking and displaying the relative position of a catheter within a user's body. In particular, described herein are methods, systems and apparatuses for tracking and displaying a real-time position of a catheter overlaying a static fluoroscopic image. The methods, systems and apparatuses disclosed herein can advantageously provide tracking of the location of a catheter and can significantly reduce radiation exposure to the patients, physicians, and staff during the interventional procedure.

The systems described herein can include a sensor for tracking the relative position of a catheter within a user's body. The sensor can be, for example, disposed at an insertion point of the catheter. Alternatively, the sensor can be attached to a distal end of the catheter. The sensor can be configured to provide a real time axial position of the catheter, such as a real time axial position of the catheter overlaying on a static image, such as a static fluoroscopic image.

The methods, systems and apparatuses disclosed herein can significantly reduce radiation exposure to the patients, physicians and staff during interventional procedures.

Examples of the types of catheters with which the sensor (and associated tracking system) may be used include: (1) guidewire support/placement catheters; (2) support/placement imaging catheters; (3) occlusion crossing catheters (4) occlusion crossing imaging catheters; (5) atherectomy catheters; and (6) atherectomy imaging catheters. Exemplary catheters with which the sensors and/or tracking may be used are described in U.S. patent application Ser. Nos. 13/433,049 and 13/939,161, the entireties of which are incorporated by reference herein.

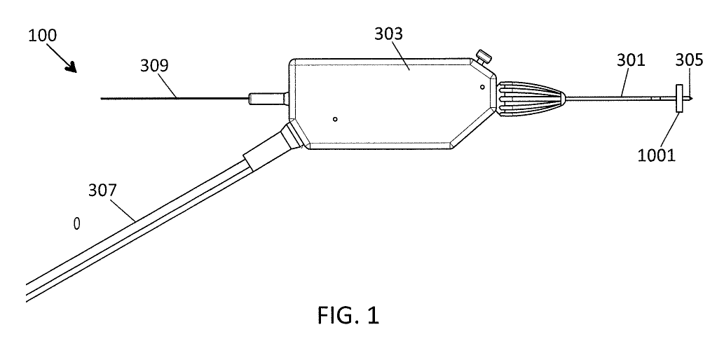

An exemplary catheter with which the tracking system may be used is shown in FIG. 1. The catheter 100, which may be used, for example, as a guidewire positioning catheter or an atherectomy catheter, can include an elongate flexible shaft 301 and a rotatable distal tip 305 having an imaging sensor, such as an OCT sensor, connected thereto. The imaging sensor at the distal tip 305 can provide imaging of the vessel structure and morphology as it is being traversed. Imaging may be forward-facing, lateral-facing, adjustable between forward-facing and lateral-facing, and/or rear-facing or angled between the forward and lateral facing. The imaging sensor can be part of an optical fiber that is fixed at one end to the distal tip 305, but is otherwise free to move around, such as within an internal lumen between a lumen housing the guidewire 309 and an outer casing of the shaft 301.

The shaft 301 can extend from a handle region 303 and terminate in the rotatable distal tip 305. A guidewire 309 can extend through the catheter device 100, such as through a guidewire lumen in or running along the side of the shaft 301. The guidewire 309 may be held in the device 100 as it is positioned within a patient or it may be inserted after the distal end of the shaft 301 has been positioned within the lumen of the vessel, such as past an occlusion or lesion.

The handle region 303 can house the control mechanism for controlling the rotation of the distal tip 305 (and OCT reflector/sensor at the end of the optical fiber). The control mechanism can control the rotation of the distal tip 305 and/or the imaging sensor attached thereto. In some embodiments, the handle region 303 can also control the rate of rotation. Power and imaging lines 307 may extend from the handle region 303 to connect the optical fiber with a power source and a light source for the imaging (e.g., OCT) system.

In some embodiments, the catheter can further have a steer mechanism built therein, such as a fixed or deflectable jog, a selective stiffening member, which may be withdrawn/inserted to help steer the device, and/or one or more tendon members to bend/extend the device for steering.

Further, the catheter 100 can include a position sensor 1001 near the distal tip 305. In one embodiment, the sensor 1001 can be an optical sensor. The optical sensor can be a light detector, such an array of photodiodes, an optoelectronic sensor, an opto-mechanical sensor, or an opto-magnetic sensor. The optical sensor can be a small camera looking at markings on the catheter shaft and tracking the movement of the shaft to infer distance. In another embodiment, the sensor 1001 can be a mechanical sensor. The mechanical sensor can be, for example, a ring around the catheter with mechanical wheels, and as the catheter 100 is moved distally or proximally, the wheels can turn and an encoder can sense the catheter travel. In other embodiments, the sensor 1001 can be an electromagnetic positioning sensor, a pressure wire configured to sense proximal or distal movement, or a voice coil sensor.

In some embodiments, the sensor 1001 is permanently mounted at the distal end 305 of the catheter 100. In other embodiments, the sensor 1001 can be movably attached to the elongate body 301 such that the sensor 1001 can remain stationary as the catheter body 1001 moves proximally and/or distally. In yet other embodiments, the sensor 1001 can be completely detached from the elongate body 301.

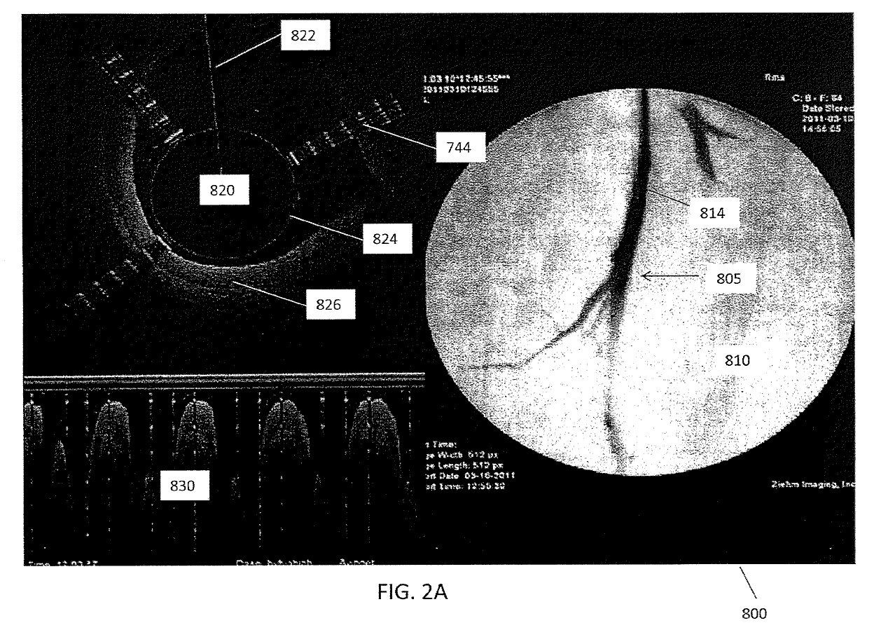

FIGS. 2A and 2B are exemplary screen captures of an imaging output associated with using a catheter such as catheter 100 in a blood vessel. In FIGS. 2A and 2B, the displayed image 800 is divided into three components. On the right is a fluoroscopic image 810 showing the distal end 805 of the catheter within a vessel 814. Contrast has been inserted into the vessel 814 to show the extent of the vessel 814 and any occluded regions.

An OCT image 820 is shown on the left. To obtain the OCT image 820, for example, the distal tip of the catheter (including an OCT sensor) rotates, and the OCT system provides a continuous set of images as the catheter rotates within the vessel. The images are combined into a continuously updated OCT image 820 that corresponds to the inside of the vessel in which the catheter is inserted. That is, the OCT image 820 is an image trace of the interior of the vessel just proximal to the distal tip as it rotates. The line 822 (extending to almost 12 o'clock in the figure) indicates the current direction of the OCT laser beam as it is rotating. The circle 824 in the middle of the image 820 represents the diameter of the catheter, and thus the area surrounding the circle 824 indicates the vessel. The OCT imaging can extend more than 1 mm from the imaging sensor, such as approximately 2 mm or approximately 3 mm, and thus will extend into the walls of the vessel (particularly in the closer region of the vessel) so that the different layers 826 of the vessel may be imaged. In this figure, the three striped rays 744 (extending at approximately 2 o'clock, between 7 and 8 o'clock, and approximately 11 o'clock) indicate the location of the three spines of the catheter and thus may act as directional markers, indicating the orientation of the distal end of the catheter within the body. As described in more detail below, the user may also be able to determine relative orientation of the OCT image (relative to the patient's body orientation) using these striped rays 744.

On the bottom left of the image 800 is a waterfall view 830 of the OCT image as it circles the radius of the body. This waterfall image 830 may be particularly useful in some applications of the system, for example, indicating the relative longitudinal position of a feature (e.g., layered structures, occlusions, branching region, etc.) as the device is moved longitudinally within the vessel. The waterfall view 830 typically includes a time axis (the x-axis) while the y-axis shows the image from the OCT sensor. In addition, the waterfall view 830 may provide an indication of when the catheter has crossed an occlusion. For example, the waterfall view 830 may show the patient's heartbeat when the walls of the vessel move relative to the heartbeat. In these cases, the waterfall view 830 may show the walls of the vessel moving with the heartbeat. In contrast, when the distal tip is within an occlusion the wall of the vessel, the waterfall view will not show movement of the walls since the occlusion material typically prevents the movement of the walls due to the heartbeat, while in healthy vessels the heartbeat is apparent. Thus it may be possible to determine when the catheter has crossed the occlusion based on the waterfall view 830. In some variations, this effect may be automated to provide an indication of when the device is within or has crossed an occlusion. In general, crossing the boundary of a total occlusion is not well defined and may result in inadvertently dissecting the vessel. When the catheter is within the true lumen, the vessel wall may move; if the catheter tip is not in the true lumen all or part of the vessel wall will not move. Thus, this movement of the wall during heartbeat may reflect the position within the true versus false lumen.

FIG. 2B shows another screen capture from the same procedure shown in FIG. 2A. As shown in the fluoroscopy image 810, the distal tip 305 is further within the vessel 814 than in FIG. 2A. In this example, the OCT image 820 shows a branch 818 of the vessel extending from the vessel in the 2 o'clock position.

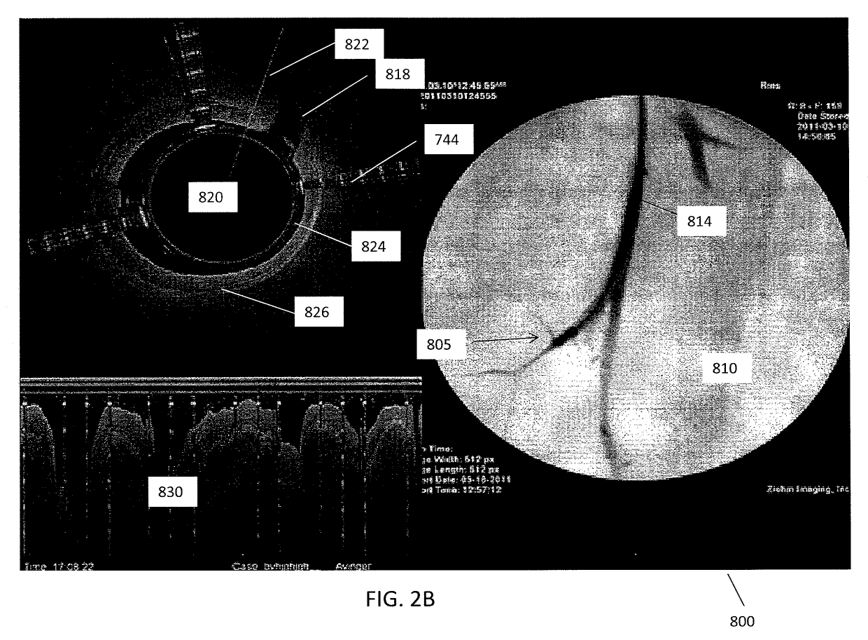

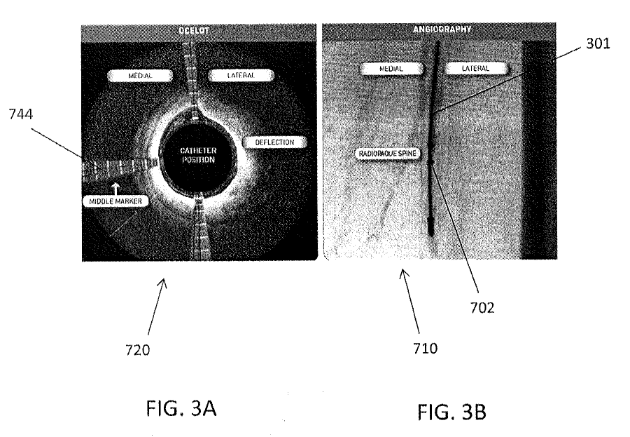

In some embodiments, the generated fluoroscopy images and OCT images can be oriented relative to one another, e.g., so that what the user sees on the right side of the OCT image is consistent with what the user sees on the right side of the fluoroscopy image. Referring to FIGS. 3A and 3B, the shaft 301 can include a fluoroscopy marker 702 that provides varying contrast in a fluoroscopy image depending on its radial orientation. The marker may be a radiopaque band with one or more asymmetric features such as a “C”, “T”, or dog bone shape that can be used to radially orient the shaft because the fluoroscopic image of the marker will change depending on its orientation. The fluoroscopy marker 702 can be used to align a fluoroscopy image 710 with an OCT image 720 during use of the catheter.

As shown in FIGS. 3A and 3B, to align the fluoroscopy image 710 with the OCT image 720, the shaft 301 can be rotated slightly such that the marker 702 is aligned to a particular side of the screen, such as at the 9 o'clock position. The up/down position of the catheter can also be determined. After the rotational position and the up/down position of the catheter have been determined using the fluoroscopy image 710, the OCT image can then be oriented such that striped ray 744 from the middle marker of the shaft 301 is also at the 9 o'clock position in the OCT image 720. Such positioning can be termed “fluorosyncing.” Fluorosyncing can be performed using manual input from the user, such as information regarding the up/down position and the rotational position, or can be performed automatically. To orient the OCT image 720 using this information, the software may draw the OCT image 720 either in a clockwise or counterclockwise direction (depending on the up/down orientation of the catheter in the fluoroscopy image 710) and will rotate the image 90°, 180°, or 270° (depending on the rotational position of the catheter in the fluoroscopy image 710).

In some embodiments, the fluoroscopic image can be used continuously and/or intermittently with the procedure to determine the position of the catheter. In other embodiments, however, the fluoroscopic image can be taken initially (e.g., upon insertion of the catheter into the body lumen) and then used as a static image over which the position of the catheter can be displaced. Advantageously, by reducing the duration of fluoroscopic imaging, the system can both be simplified and the patient's exposure to x-ray radiation can reduced.

Thus, referring back to FIG. 1, the sensor 1001 can be used to identify the relative position of the catheter 100. That is, the sensor 1001 can provide information regarding the real time position of the catheter 100, which can be overlaid on a static fluoroscopic image to guide an interventional procedure. In some embodiments, the OCT image and the real time position of the catheter overlaying the static fluoroscopic image can be shown on a same display for convenient viewing by the physicians to guide the interventional procedure.

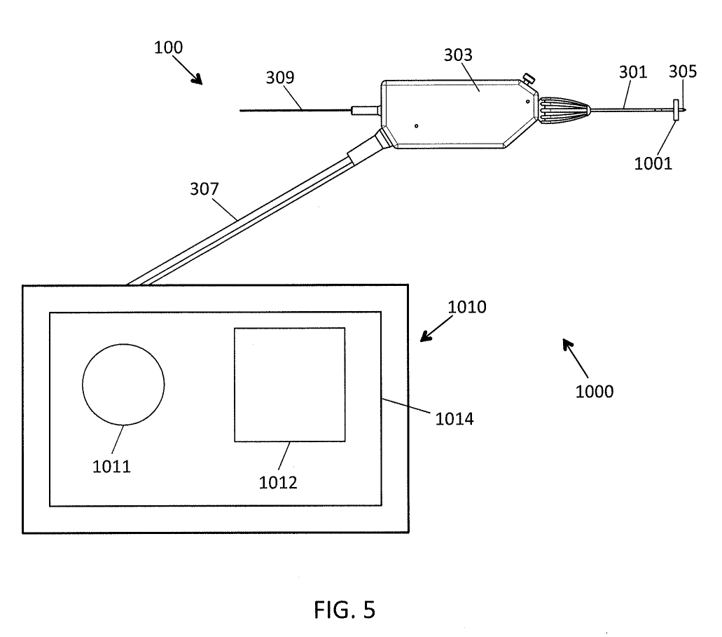

FIG. 5 illustrates a block diagram of a system 1000 configured to track and display a real time position of the catheter 100. The system 1000 includes a processor 1010 having a display 1014. The display 1014 can include a static image 1012 of the portion of the body in which the catheter is inserted (e.g., a static fluoroscopic image) and an OCT image 1011 gathered from the OCT sensor on the catheter 100. The longitudinal insertion distance of the catheter 100 can be measured by the sensor 1001 and displayed on the static image 1012 simultaneously with the display of the OCT image 1011. The processor 1010 can be configured to receive signals from the sensor 1001 and use those signals to determine a position of the catheter 1001. For example, the processor 1010 can be configured to translate pixels into distance when an optical sensor is used. The display 1014 can be configured to display the position of the catheter 1001 overlaying a static image 1012. For example, the processor 1010 can be configured to translate a distance displacement into a scaled drawing on the display 1014.

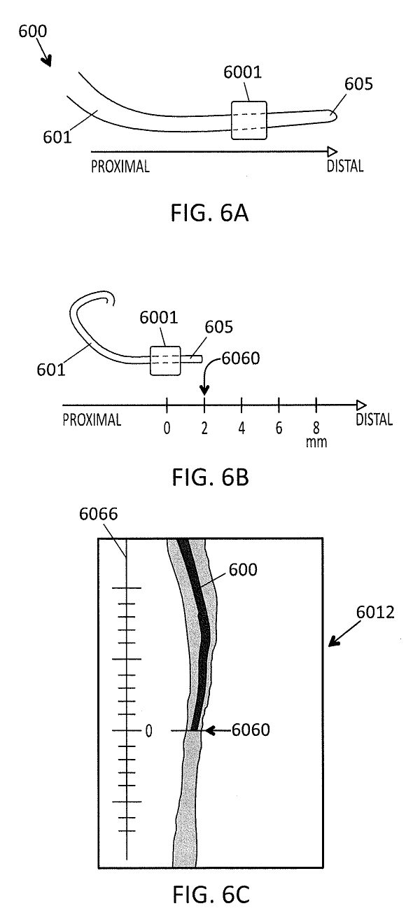

FIGS. 6A-8B show one exemplary method of tracking the position of a catheter. As shown in FIG. 6A, the catheter 600 can include a catheter body 601, sensor 6001 and distal tip 605. In this embodiment, the catheter body 601 can be configured to translate relative to the sensor 6001. For example, the sensor 6001 can include a mechanical mount disposed at the insertion point on the patient and/or the sensor 6001 can be movably attached to the catheter body 601. As shown at FIGS. 6B-6C, a “zero position” 6060 of the catheter 600 can be established by taking an image (e.g., fluoroscopic image) of the catheter after insertion into the body (e.g., into the blood vessel). The position of the distal tip 605 during the initial imaging can be considered the “zero” (or initial) position 6060 relative to the sensor 6001. In some embodiments, the fluoroscopic image 6012 can, for example, include a ruler 6066 on the image 6012 to indicate distance traveled.

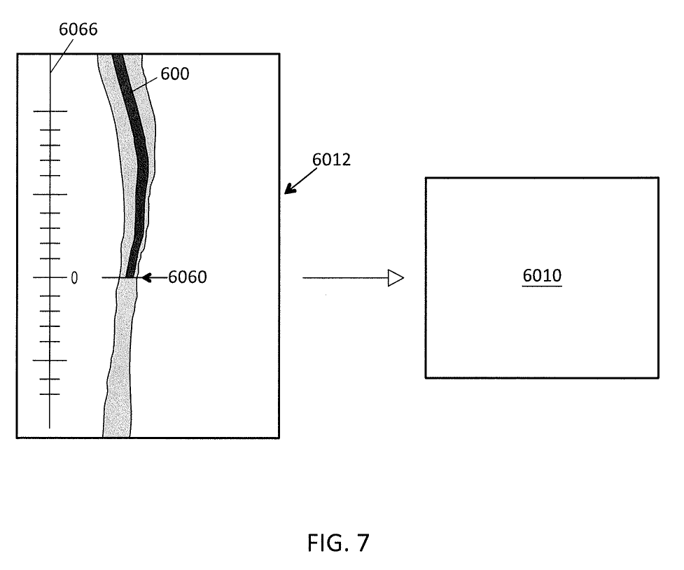

As shown at FIG. 7, the “zero” position of the catheter 600 can then be input into the processor 6010, such as a processor described in U.S. patent application Ser. Nos. 13/433,049 and 13/939,161. The zero position 6060 can be input into the processor 6010 by video input, or Digital Imaging and Communications in Medicine (DICOM), for example.

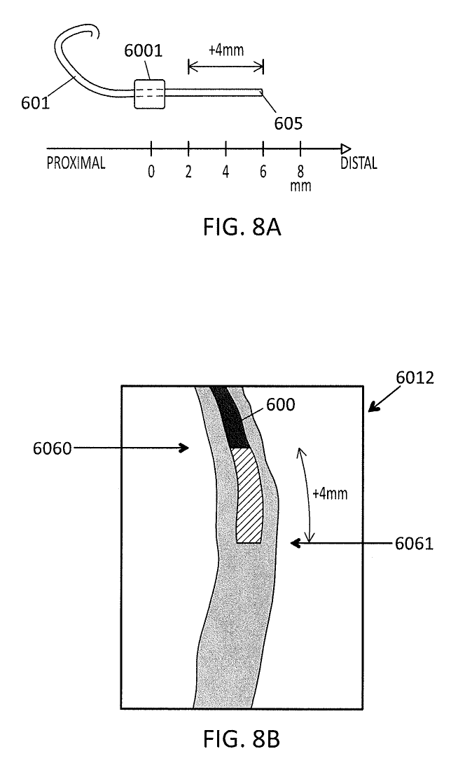

As shown at FIGS. 8A-8B, the elongate body 601 can then be advanced relative to the sensor 6001 to a second position (e.g., advanced further distally into the blood vessel). The sensor 6001 can be configured to measure displacement of the elongate body 601 and/or distal tip 605 relative to the sensor 6001. For example, the sensor 6001 can gather optical images, electrical signal, or electro-magnetic signals to determine the position. In some embodiments, the sensor 6001 can be an electrical sensor, and the relative displacement between the elongate body 601 and the sensor 6001 can result in a voltage change. The sensor 6001 can then send the gathered signals to the processor, which can determine the displacement. For example, when the sensor 6001 is an optical sensor, the processor can be configured to translate the number of pixels into physical distance to measure the displacement of the catheter. The processor can be further configured to translate distance measured by the sensor to a distance or displacement drawn on the image 6012, as shown in FIG. 8B. That is, as shown in FIG. 8B, the estimated position 6061 of the catheter 600 can be displayed on the static image 6012. In some embodiments, the relative displacement of the distal end 605 of the catheter from the zero position 6060 to the second position 6061 can be indicated by a different color or shading on the static image 6012.

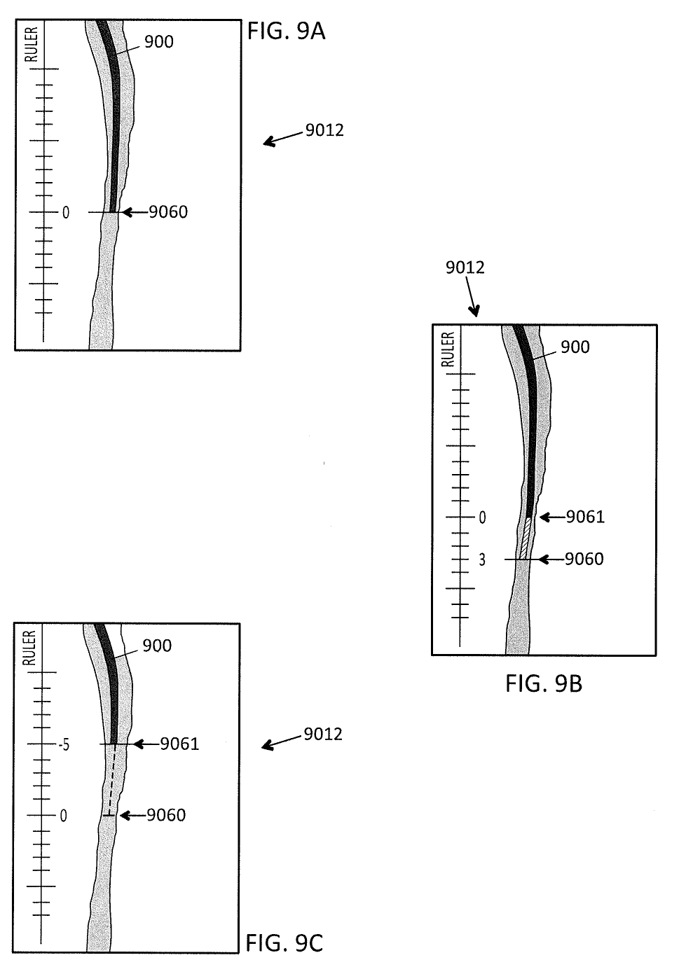

FIGS. 9A-9C schematically illustrate different measured positions 9061 of the distal tip of a catheter 900 relative to a zero position 9060 on a static image 9012 (e.g., a static fluoroscopic image). FIG. 9A schematically illustrates the catheter 900 at the zero position 9060 (i.e., when the fluoroscopic image is captured). FIG. 9B illustrates the catheter 900 advanced further distally inside a vessel of a patient relative to the zero position 9060 by 3 units (e.g., 3 cm) to a second position 9061. FIG. 9C illustrates the catheter 900 retracted proximally from the zero position 9060 by a distance of 5 units (e.g., 5 cm) to a second position 9061.

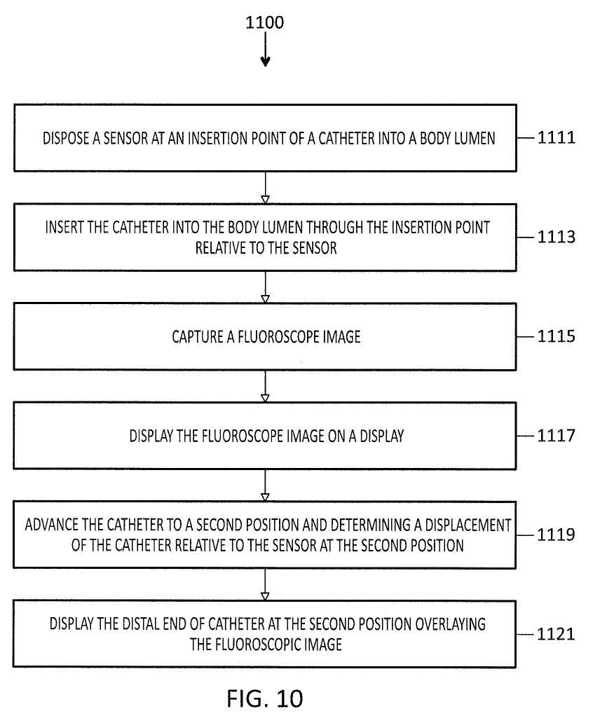

FIG. 10 illustrates a flow diagram of a method 1100 for tracking and displaying a real-time catheter position overlaying a fluoroscopic image. At step 1111, a sensor can be disposed at an insertion point of a catheter into a body lumen. At step 1113, the catheter can be inserted into the body lumen through the insertion point. At step 1115, a fluoroscopic image can be captured that indicates the position of the distal end of the catheter at a first position. At step 1117, the fluoroscopic image can be displayed on the processor display. At step 1119, the catheter can be advanced to a second position. The displacement of the catheter relative to the sensor can be determined. At step 1121, the catheter at the second position can be displayed as an overlay on the fluoroscopic image on the display.

The method can include synchronizing a zero position of the distal end of the catheter with the first position when the fluoroscopic image is captured. For example, the method can include displaying the zero position of the distal end of the catheter on the fluoroscopic image as shown in FIGS. 9A-9C. The method can include determining the displacement of the catheter comprising determining the location of the distal end of the catheter at the second position by signals from the sensor. For example, various sensors can be used to measure the displacement. The method can further include only capturing a single fluoroscopic image for a travel range of the catheter that is displayed within a view of the single fluoroscopic image, thus significantly reduce an overall amount of x-ray radiation during an interventional procedure.

The systems and methods disclosed herein can advantageously track and display a real time position of the catheter inside the vessel of the patient to guide an interventional procedure and significantly reduce x-ray radiation. Instead of continuous x-ray radiation as in conventional fluoroscopy, the systems and methods disclosed herein only need to take a fluoroscopic image once, as long as the catheter is within a travel range that can be displayed in the view of the fluoroscopic image. For example, if the length of the fluoroscopic image is 10 cm, and the catheter's initial position is at a “1 cm” mark, the catheter can have a travel range of 9 cm before it is out of view. Once the catheter is being advancing farther, another fluoroscopic image may be taken. By this way, the amount of radiation can be significantly reduced.

The catheters described herein can be dimensioned to fit within lumens of the body, such as blood vessels. For example, the catheters can be configured to be placed within the peripheral blood vessels. Thus, the catheters can have an outer diameter of less than 0.1 inch, such as less than 0.09 inches, such as less than or equal to 0.08 inches.

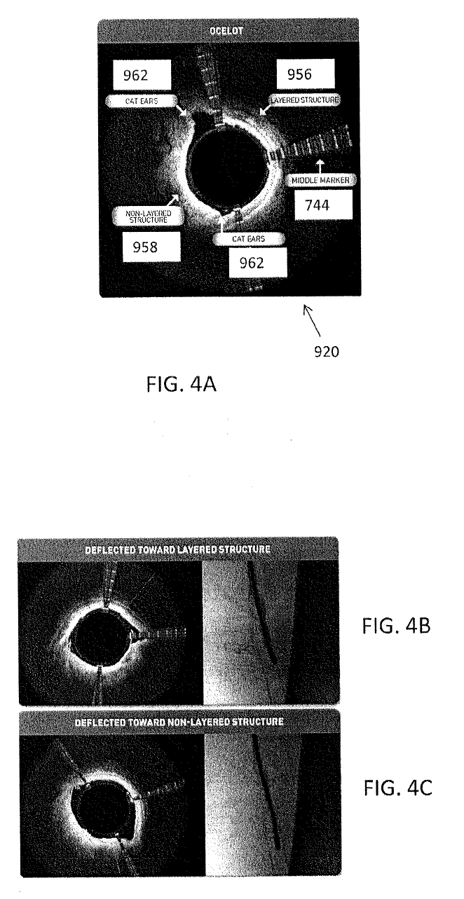

Further, the methods and systems described herein can be used to orient the catheter in the desired direction and/or to move the catheter to the desired location. Referring to FIG. 4A, the OCT image 920 shows healthy tissue 956 in the form of a layered structure and non-healthy tissue 958 in the form of a nonlayered structure. The cat ears 962 in the image show a region between the healthy and unhealthy tissue caused by a slight expansion of the vessel around the catheter at that location. Accordingly, during an OCT procedure, one goal may be to steer the catheter towards the unhealthy tissue. FIG. 4B shows the catheter deflected toward the layered, healthy tissue. FIG. 4C shows the catheter rotated such that it is deflected toward the unhealthy, non-layered structure.

In some embodiments, for catheters with an optical coherence tomography (OCT) sensor, for catheters with an optical coherence tomography (OCT) sensor, the real time position of the catheter overlaid on a static fluoroscopic image can be displayed next to the OCT image. This can advantageously add a third dimension of perspective that a simple OCT imaging system may lack. The longitudinal position of the catheter can allow encoding each OCT image with a position value that is required for a 3D volume reconstruction. A stack of OCT images with a position for each image can be used to “stitch” together and render a 3D volume of the imaged region. Because only a single fluoroscopic image is required to be captured for a travel range of the catheter that can be displayed in the view of the fluoroscopic image, the overall radiation time can be significantly reduced.

In some embodiments, the sensors and systems described herein can be configured to work with a catheter device without an OCT imaging sensor. For catheters without an OCT sensor, the distal end of the catheter overlaying the fluoroscopic image or a position of the distal end of the catheter can be displayed to guide a surgical procedure.

It should be understood that any element described herein with respect to one embodiment can be combined or substituted for any element described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for tracking and displaying a real-time catheter position overlaying a fluoroscopic image, the system comprising:
a catheter having an optical coherence tomography (OCT) sensor thereon;
a displacement sensor configured to identify displacement of the catheter; and
a controller configured to:
receive a fluoroscopic image of the distal end of the catheter at a first position; and
determine a displacement of the catheter from the first position to a second position using the displacement sensor; and
a display configured to display the distal end of catheter at the second position as an overlay on the fluoroscopic image.

2. The system of claim 1, wherein the display is further configured to display an OCT image from the OCT sensor.

3. The system of claim 1, wherein the fluoroscopic image is a static fluoroscopic image.

4. The system of claim 1, wherein the controller is further configured to synchronize a zero position of the distal end of the catheter with the first position when the fluoroscopic image is captured.

5. The system of claim 1, wherein determining the displacement of the catheter comprises determining the location of the distal end of the catheter at the second position using signals from the displacement sensor.

6. The system of claim 1, wherein the displacement sensor is attached to the catheter.

7. The system of claim 6, wherein the displacement sensor is axially movable relative to the catheter.

8. The system of claim 1, wherein the displacement sensor is a separate component from the catheter.

9. The system of claim 1, wherein the displacement sensor is an optical sensor.

10. The system of claim 1, wherein the displacement sensor is a mechanical sensor.

11. The system of claim 1, wherein the displacement sensor is an electromagnetic positioning sensor.

12. The system of claim 1, wherein the catheter is an atherectomy catheter.

13. The system of claim 1, wherein the catheter is an occlusion-crossing catheter.

14. A method for tracking and displaying a real-time catheter position comprising:
inserting a catheter into a body lumen;
capturing an optical coherence tomography (OCT) image with an OCT sensor on the catheter;
capturing a fluoroscopic image of the distal end of the catheter at a first position;
displaying the fluoroscopic image on a display;
advancing the catheter to a second position;
determining a displacement of the catheter from the first position to the second position using a displacement sensor; and
displaying the distal end of catheter at the second position as an overlay on the fluoroscopic image on the display.

15. The method of claim 14, wherein capturing a fluoroscopic image comprises capturing a static fluoroscopic image, and wherein displaying comprises displaying the static fluoroscopic image.

16. The method of claim 14, further comprising synchronizing a zero position of the distal end of the catheter with the first position when the fluoroscopic image is captured.

17. The method of claim 16, further comprising displaying the zero position of the distal end of the catheter on the fluoroscopic image.

18. The method of claim 14, wherein determining the displacement of the catheter comprises determining the location of the distal end of the catheter at the second position using signals from the displacement sensor.

19. The method of claim 14, wherein the method further includes displaying the OCT image.

20. The method of claim 19, further comprising displaying the distal end of the catheter overlaying the fluoroscopic image on a same display as the OCT image.

21. The method of claim 14, wherein the displacement sensor is attached to the catheter.

22. The method of claim 21, wherein the displacement sensor is axially movable relative to the catheter.

23. The method of claim 14, further comprising placing the displacement sensor at an insertion point of the catheter into the body lumen.

24. The method of claim 14, further comprising reducing an overall amount of x-ray radiation by only capturing a single fluoroscopic image for a travel range of the catheter that is displayed within a view of the single fluoroscopic image.

25. The method of claim 14, wherein the displacement sensor is an optical sensor.

26. The method of claim 14, wherein the displacement sensor is a mechanical sensor.

27. The method of claim 14, wherein the displacement sensor is an electromagnetic positioning sensor.

28. A catheter device comprising
an elongate body extending from a proximal end to a distal end;
an optical coherence tomography (OCT) sensor attached to the elongate body; and
a displacement sensor attached to the elongate body and axially movable relative to the movable body, the displacement sensor configured to measure a displacement of the distal end of the catheter relative to the displacement sensor;
wherein the displacement sensor is configured to be connected to a controller configured to receive signals from the displacement sensor and determine a position of the distal end.

29. The catheter device of claim 28, wherein the displacement sensor is an optical sensor.

30. The catheter device of claim 28, wherein the displacement sensor is a mechanical sensor.

31. The catheter device of claim 28, wherein the displacement sensor is an electromagnetic positioning sensor.

32. The catheter device of claim 28, wherein the catheter is an atherectomy catheter.

33. The catheter device of claim 28, wherein the catheter is an occlusion-crossing catheter.
